(19) United States
(12) Patent Application Publication
(10) Pub. No.: US 2009/0138096 A1
(43) Pub. Date: May 28, 2009

(54) FOOT/ANKLE IMPLANT AND ASSOCIATED METHOD

(76) Inventors: Mark S. Myerson, Baltimore, MD (US); Paul J. D'Antonio, Morristown, NJ (US); Lisa C. Thompson, Riegelsville, PA (US); John Sharobiem, Freehold, NJ (US); Joseph M. Hernandez, Torrance, CA (US)

Correspondence Address: HARNESS, DICKEY & PIERCE, P.L.C., P.O. BOX 828, BLOOMFIELD HILLS, MI, 48303, US

(21) Appl. No.: 12/166,382

(22) Filed: Jul. 2, 2008

Related U.S. Application Data
(63) Continuation of application No. 11/504,271, filed on Aug. 15, 2006, now abandoned.
Application No. 11/504,271 is a continuation-in-part of application No. 11/008,075, filed on Dec. 9, 2004.
(60) Provisional application No. 60/708,820, filed on Aug. 16, 2005.
(60) Provisional application No. 60/634,448, filed on Dec. 8, 2004.

(51) Int. Cl. A61F 2/66; A61B 19/00

(52) U.S. Cl. 623/54; 128/898

(57) ABSTRACT

A foot/ankle implant anatomically-shaped for implantation between two bone portions of the foot or ankle to correct associated deformities. The foot/ankle implant has a peripheral wall surrounding a central bore therethrough and defining an annular cross-section. The wall is constructed from a composite material that includes a ceramic component and a polymer component. The ceramic component is gradually resorbed after implantation, and the polymeric component gradually degrades after implantation.

### CROSS-REFERENCE TO RELATED APPLICATIONS

This is a continuation of U.S. patent application Ser. No. 11/504,271, filed on Aug. 15, 2006, which claims the benefit of U.S. Provisional Application No. 60/708,820, filed on Aug. 16, 2005.

This application is also a continuation-in-part of U.S. patent application Ser. No. 11/008,075, filed on Dec. 9, 2004, which claims the benefit of U.S. Provisional Application No. 60/634,448, filed on Dec. 8, 2004.

The disclosures of the above applications are incorporated herein by reference.

### INTRODUCTION

Various surgical procedures and prosthetic devices are known for the correction of foot/ankle disorders and/or deformities. Current reconstructive procedures include intra-operative shaping of autogenous bone tissue or human allograft bone tissue. Other bone grafting procedures include packing a void with a granular and/or putty-like material. Intra-operative shaping is a time-consuming process, and further the bone tissue used has limited size and shaping potential. The alternative of packing with granular and/or putty-like materials may not provide adequate structural support.

Although the existing procedures and implants for foot/ankle applications can be satisfactory for their intended purposes, there is still a need for implants that provide structural support as well as size and shape versatility for various foot/ankle procedures.

### SUMMARY

The present teachings provide an orthopedic device for a foot/ankle implant. The foot/ankle implant comprises a composite structure having a ceramic component with macroporosity and a polymer component filling the macroporosity. The composite structure forms an anatomically-shaped and load-bearing graft for implantation between two bone portions of the foot or ankle to correct associated deformities. The ceramic component is gradually resorbable after implantation, the polymeric component is gradually degradable after implantation and the composite structure is gradually replaceable by tissue/bone ingrowth.

In another aspect, the present teachings provide a foot/ankle implant anatomically-shaped for implantation between two bone portions of the foot or ankle to correct associated deformities. The foot/ankle implant has a peripheral wall surrounding a central bore therethrough and defining an annular cross-section. The wall is constructed from a composite material that includes a ceramic component and a polymer component. The ceramic component is gradually resorbed after implantation, and the polymeric component gradually degrades after implantation.

The present teachings provide a method for correcting foot/ankle deformities. The method includes providing a resorbable polymer-reinforced ceramic composite block, shaping the composite block to an anatomically-shaped and load-bearing graft for implantation between two bone portions of the foot or ankle to correct associated deformities, maintaining an opening between the two bone portions before inserting the implant, and inserting the implant in the opening such that the implant substantially matches the cross-section of the bone portions. Shaping of the composite block includes pre-operative or intra-operative shaping.

Further areas of applicability of the present invention will become apparent from the detailed description provided hereinafter. It should be understood that the detailed description and specific examples are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is a perspective view of a foot/ankle implant according to the present teachings;

FIG. 2 is a perspective view of a foot/ankle implant according to the present teachings;

FIG. 3 is a perspective view of a foot/ankle implant according to the present teachings;

FIG. 4 is a perspective view of a foot/ankle implant according to the present teachings;

FIG. 5 is a perspective view of a foot/ankle implant according to the present teachings;

FIG. 6 is a perspective view of the foot/ankle implant of FIG. 5 shown in an environmental view indicating the location of implantation;

FIG. 7 is radiographic view of the foot/ankle implant of FIG. 5 after implantation;

FIGS. 8-10 are environmental views illustrating a method of implantation of the foot/ankle implant of FIG. 5 according to the present teachings;

FIG. 11 is a perspective view of a foot/ankle implant according to the present teaching;

FIG. 12 is side view of the foot/ankle implant of FIG. 11;

FIG. 13 is a perspective view of the foot/ankle implant of FIG. 11 shown in an environmental view indicating the location of implantation;

FIG. 14 is radiographic view of the foot/ankle implant of FIG. 11 shown after implantation;

FIGS. 15 and 16 are environmental views illustrating a method of implantation of the foot/ankle implant of FIG. 11 according to the present teachings;

FIG. 17 is a perspective view of a foot/ankle implant according to the present teachings;

FIG. 18 is a plan view of the foot/ankle implant of FIG. 17;

FIG. 19 is a perspective view of the foot/ankle implant of FIG. 17 shown in an environmental view indicating the location of implantation;

FIG. 20 is radiographic view of the foot/ankle implant of FIG. 17 shown after implantation;

FIGS. 21 and 22 are environmental views illustrating a method of implantation of the foot/ankle implant of FIG. 17 according to the present teachings;

FIG. 23 is a perspective view of a foot/ankle implant according to the present teachings;

FIG. 24 is a perspective view of the foot/ankle implant of FIG. 23 shown in an environmental view indicating the location of implantation;

FIG. 25 is radiographic view of the foot/ankle implant of FIG. 23 shown after implantation;

FIGS. 26 and 27 are environmental views illustrating a method of implantation of the implant of FIG. 23 according to the present teachings;

FIGS. 28A and 28B are schematic illustrations of fastening devices optionally associated with various foot/ankle implants according to the present teachings;

FIG. 29A is a perspective view of a foot/ankle implant according to the present teachings;

FIG. 29B is a plan view of the foot/ankle implant of FIG. 29A;

FIG. 29C is a perspective view of a foot/ankle implant according to the present teachings;

FIG. 30A is a perspective view of a foot/ankle implant according to the present teachings;

FIG. 30B is a plan view of the foot/ankle implant of FIG. 30A;

FIG. 30C is a sectional view of the foot/ankle implant of FIG. 30B taken along axis 30C;

FIGS. 31A and 31B are perspective views of utility blocks according to the present teachings; and

FIG. 32 is a perspective view of a foot/ankle implant according to the present teachings.

### DESCRIPTION OF VARIOUS ASPECTS

The following description is merely exemplary in nature and is in no way intended to limit the invention, its application, or uses. For example, although the present teachings are illustrated for specific foot or ankle procedures, such as, for example, calcaneal osteotomies, subtalar fusions, cuneiform osteotomies, and hallux metatarsal-phalangeal fusions, the present teachings can be used for other foot/ankle grafts that are not specifically illustrated, such as various ankle fusions, supramaleolar osteotomies, and other graft procedures. Further, it should be noted that the foot/ankle implants can be implanted between two bone portions formed by an osteotomy procedure of a single bone, or between two separate bones, such as in the space between articulating or otherwise contacting bones, with or without resection of the articulating/contacting surfaces.

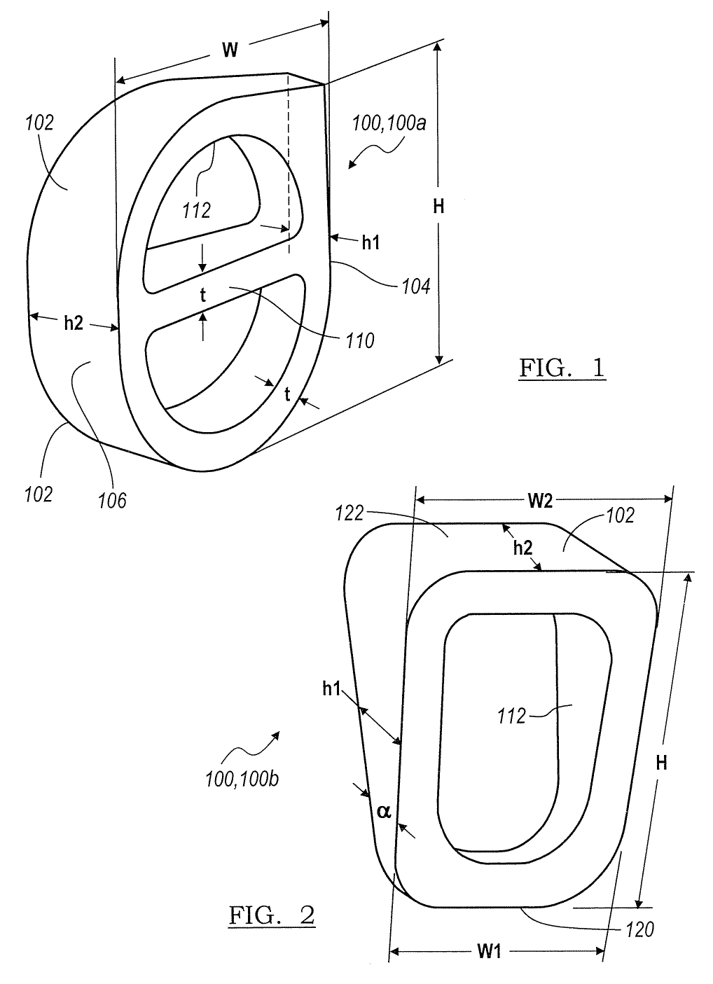

Referring to FIGS. 1-4, various exemplary anatomically-shaped foot/ankle implants 100 are illustrated according to the present teachings. Each foot/ankle implant 100 comprises a precision-made anatomical construct that is designed and pre-constructed for implantation in a particular anatomic location of the foot or ankle. Each foot/ankle implant 100 can be constructed from material that, at least in its final form, can be precision-machined to a desirable shape and/or size. Examples of such materials include, but not limited to, human bone, bovine bone, porcine bone, any calcium salt, any resorbable polymer (such as polylactic acid, polyglycolic acid, polycaprolactone, or any blend thereof), any calcium salt/polymer composite, polyetheretherketone (PEEK), PEEK/carbon fiber composite, and any of these materials loaded with a biologic agent, such as, for example, a growth factor, a peptide, an antibiotic, or any other biologic agent.

The foot/ankle implants 100 can also be constructed from a continuous phase ceramic/polymer composite, such as the composite disclosed and described in co-pending and co-assigned U.S. patent application Ser. No. 11/008,075, filed on Dec. 9, 2004. The disclosures of the U.S. patent application Ser. No. 11/008,075 are incorporated herein by reference. The composite is commercially available under the trade name BioPlex and includes a resorbable ceramic component as a base material, such as Pro Osteon® 500R. Both BioPlex and Pro Osteon® 500R are commercially available from Interpore Cross International, Irvine, Calif. Pro Osteon® is a coral-derived calcium carbonate/hydroxyapatite porous material. The macroporosity of Pro Osteon® can be filled with a second component, such as a poly(L-lactide-co-D,L-lactide) (PLDLLA) or other polymeric material using injection molding or other procedure. Pro Osteon® has a fully interconnected, porous structure that allows polymer penetration through its entire macroporosity. Pro Osteon® comprises a thin layer of hydroxyapatite over a calcium carbonate skeleton. Although the large pores within Pro Osteon® are filled with the polymer, small nanopores within the ceramic region can be maintained. These nanopores do not allow for bone in-growth, but they do allow for the transport of water and degradation products throughout the composite, thereby preventing building up of pockets of acidic monomer. Accordingly, the resulting composite is a biocompatible material that can be machined or otherwise processed to provide precision implants characterized by structural integrity. Further, and after implantation, the ceramic component of the composite is gradually resorbable, the polymeric component is gradually degradable, and the composite is gradually replaceable by tissue/bone ingrowth.

More specifically, once implanted, the Pro Osteon® component/phase is gradually resorbed by osteoclasts allowing bone and blood vessels to penetrate into the center of the implant wall, and not just to particles exposed at the surface, as is the case with particulate composites. The polymer phase is gradually broken down into soluble lactic acid by-products and carried away/removed from the implantation site. Accordingly, tissue and bone can grow throughout the entire composite implant and gradually replace the resorbed or degraded portions of the implant.

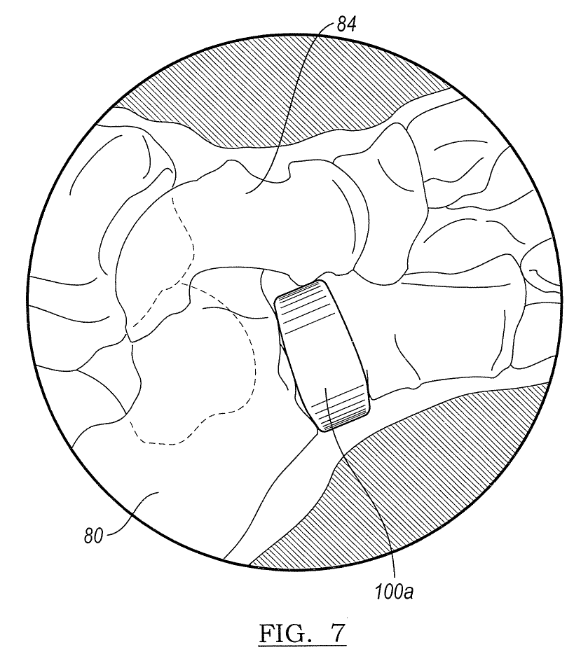

Referring to FIGS. 1 and 5-10, a precision implant 100a configured as an anatomically-shaped graft for calcaneal osteotomy for lateral column lengthening is illustrated. The precision implant 100a can be used, for example, to correct varus and arch deformities. The precision implant 100a can be wedge-shaped having a leading edge 104, which is inserted first, and a trailing edge 106. Referring to FIG. 6, the associated surgical procedure is an opening wedge osteotomy of the lateral column of the calcaneus 80 to correct arch or varus angle deformities of the foot. A lateral approach can be used to expose the calcaneus 80, as illustrated in FIG. 6. The osteotomy can be created by an appropriate instrument, such as a reciprocating saw 150, as illustrated in FIG. 9. The opposite surfaces 151 of the calcaneous bone portions created by the osteotomy can be pulled apart to form an osteotomy opening 152 using a laminar spreader or other appropriate instrument 154, as illustrated in FIG. 9, in the direction indicated by arrows “A”. The osteotomy opening 152 can be a sufficiently large, wedge-shaped opening for receiving the precision implant 100 without forcing the precision implant 100a against the opposite bone surfaces 151. The precision implant 100a can then be inserted into the osteotomy opening 152 which is maintained in a desired wedge configuration by the spreader 154 between the two bone portions of the calcaneus 80, as illustrated in FIG. 10. After the spreader 154 is removed, the opposite bone surfaces 151 move in the indicated by arrows “B” to wedge the precision implant 100a therebetween. In this procedure, any change in the relative orientation/alignment of the cut bone portions of the calcaneous 80 is effected and maintained by the spreader 154 before implantation. After implantation and removal of the spreader 154, the relative orientation of the bone portions is maintained by the precision implant 100a. A drawing of a radiographic view showing the precision implant 100a wedged into the osteotomy opening 152 is illustrated in FIG. 7.

The precision implant 100a can be configured to anatomically match the cross-section of the lateral column of the calcaneus 80 for optimal graft/host interface. More specifically, the precision implant 100a can have a generally oval or other closed curve cross-section, comprising a plurality of arcs 102 with varying radii of curvature. In one particular and exemplary aspect, the height H of the cross-section of the precision implant 100a can be about 23 mm, and the width W of the cross-section about 20 mm. The leading edge 104 of the precision implant 104a can have a leading edge elevation h₁ of about 3 mm. The magnitude of the elevation h₁ can be selected based on the particular osteotomy to be performed. The 3 mm elevation, for example, can be appropriate for an osteotomy performed in the lateral column, which is usually cut completely through the calcaneus 80. The generally curved or oval-shaped cross-section of the precision implant 100a and the specifically selected dimensions allow the load bearing portion of the precision implant 100a to be aligned with the cortex of the lateral column of the calcaneus 80 to reduce the risk of graft subsidence, which reduces the effectiveness of the opening wedge procedure.

Furthermore, the precision implant 100a can be provided in different shapes and sizes, thereby allowing the surgeon to select a particular size and control the degree of correction. For example, the degree of correction can be provided in three different sizes corresponding to different wedge elevations h₂ at the trailing edge 106. The trailing edge elevations h₂ can be, for example, about 9 mm, about 10.5 mm, and about 12 mm. The thickness “t” of the precision implant 100a can be about 3 mm, or any other adequate value selected for mechanical strength and for generating enough surface area to reduce graft subsidence. The precision implant 100a can be generally annular including a non-load-bearing central bore 112. In one aspect, the precision implant 100a can also includes a crossbar 110 of desired thickness t along a center axis of the precision implant 100a for structural reinforcement during implantation. The crossbar 110 divides the central bore 112 into separate sub-bores, as illustrated in FIG. 1. It will be appreciated that additional crossbars 110 can be provided, if desired. The central bore 110 and/or its sub-bores allow tissue in-growth and can be additionally packed with known growth promoting materials, including bone chips or particles, demineralized bone powder, collagen, and other osteogenic or osteoinducing compositions and biologic agents.

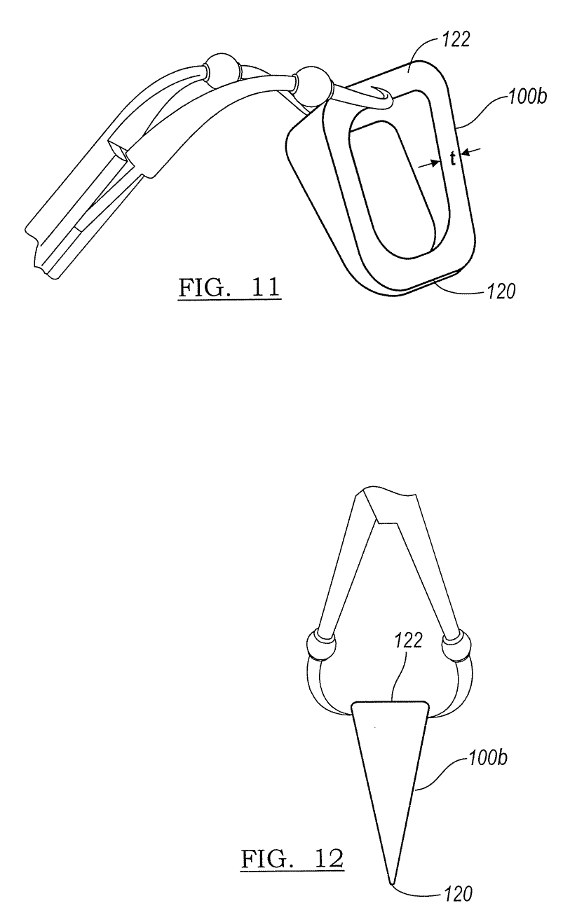

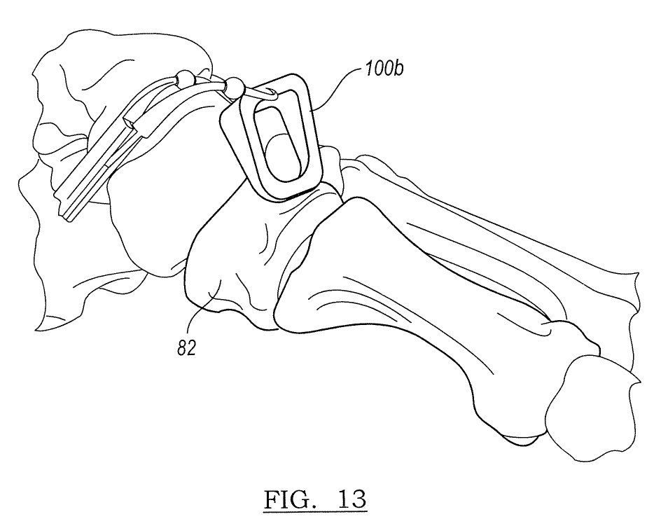

Referring to FIGS. 2 and 11-16, a precision implant 100b configured as an anatomically-shaped graft for cuneiform osteotomy is illustrated. This surgical procedure is performed on the medial cuneiform 82 to correct arch deformities, such as, for example, flatfoot deformity. The precision implant 100b can be configured as an opening wedge having a leading edge 120 and a trailing edge 122. The precision implant 100b can be provided in various sizes for different amounts of correction. The precision implant 100b can be provided, for example, with three different trailing edge elevations h₂, such as, for example, about 5 mm, about 6.5 mm, and about 8 mm, corresponding to three different wedge angles α, or other desired sizes. The precision implant 100b can be configured such that it matches the cross-section of the medial cuneiform 82 and extends approximately two-thirds of the depth of the medial cuneiform 82. The leading edge 120 of the precision implant 100b can have negligible elevation, substantially coming to a point (on a side view), as illustrated in FIG. 12, when the medial cuneiform 82 is not completely cut through during the osteotomy procedure, as is typically the case. The precision implant 100b can have a wall thickness “t” of about 3 mm, or other thickness chosen for mechanical strength and for generating enough surface area to reduce graft subsidence.

The cross-section of the precision implant 100b can be generally trapezoidal. The width W₂ of the trailing edge 122 that forms the top base of the trapezoid can be, for example, about 16 mm. The width W₁ of the leading edge 120 that forms the bottom base of the trapezoid can be, for example, about 12 mm. The height H of the trapezoidal cross-section can be about 25 mm. It will be appreciated that other dimensions can be selected, such that the precision implants 100b can have the same overall dimensions with different wedge angles, or different dimensions and different wedge angles. The cross-section of the precision implant 100b can be designed such that it will allow the load bearing portion of the precision implant 100b to be lined up with the cortex of the medial cuneiform 82 to eliminate the risks of graft subsidence and associated reduction of the effectiveness of the opening wedge procedure. The precision implant 100b can also have a non-load-bearing central bore 112 for tissue ingrowth.

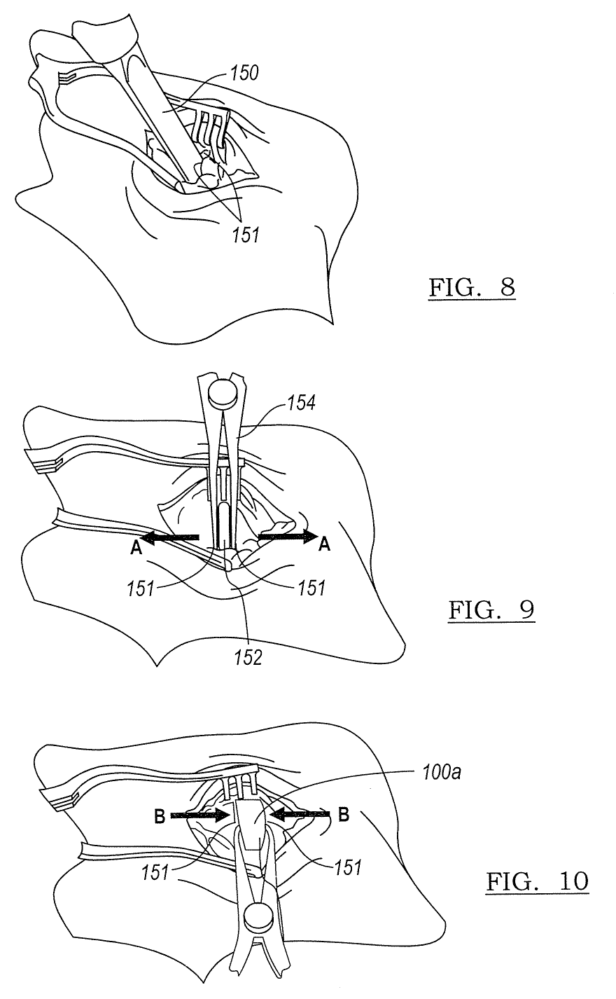

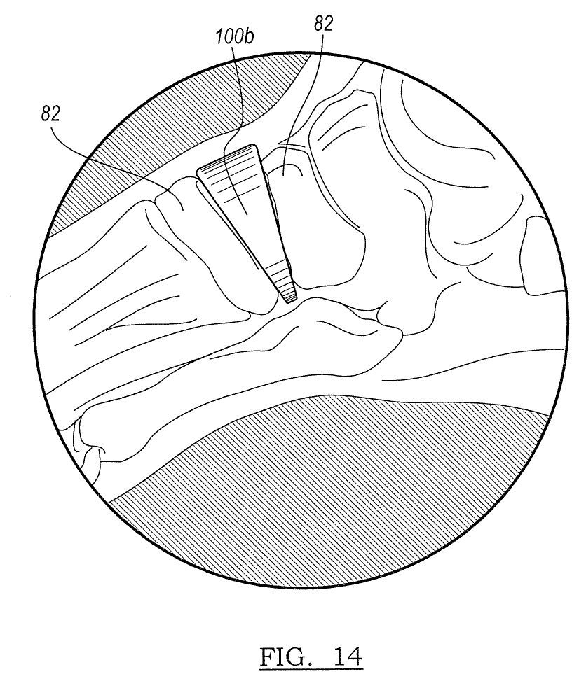

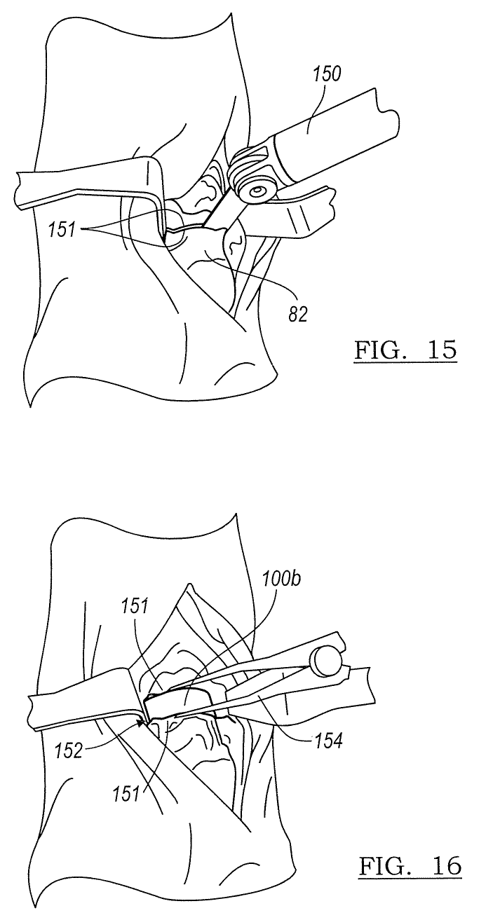

Referring to FIG. 15, an osteotomy of the medial cuneiform 82 to correct an arch deformity is illustrated using a reciprocating saw 150 forming two opposite bone the surfaces 151. Referring to FIG. 16, the precision implant 100b is shown implanted into the osteotomy opening 152 between the two bone portions 151 of the medial cuneiform 82. As described in connection with the calcaneal osteotomy illustrated in FIGS. 8-10, the osteotomy opening 152 in the cuneiform 82 is pried apart using the spreader 154 before inserting the precision implant 100b. A drawing of a radiographic view showing the precision implant 100b wedged into the osteotomy opening 152 is illustrated in FIG. 14.

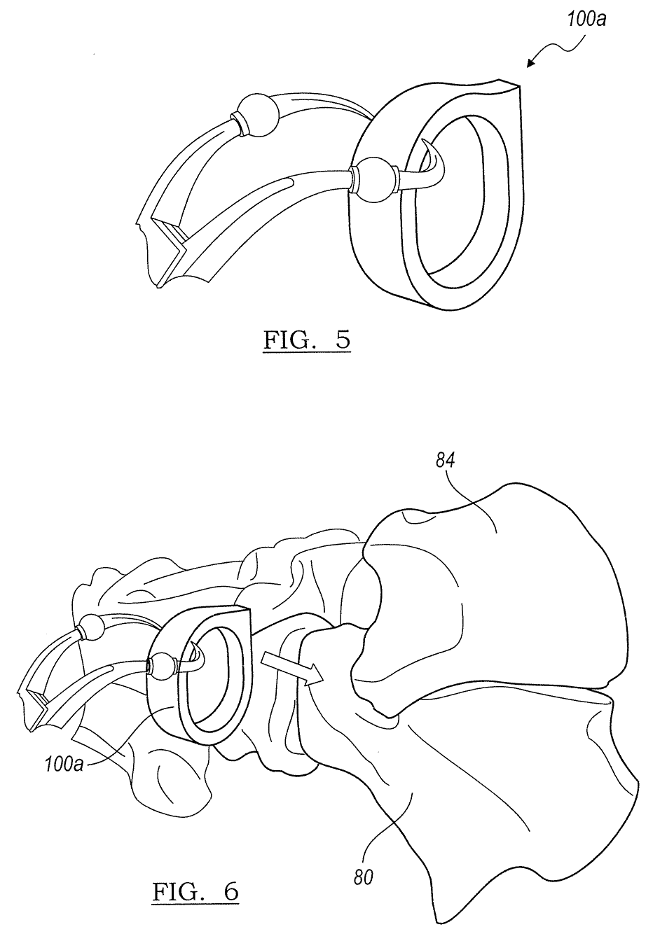

Referring to FIGS. 4 and 17-22 a precision implant 100d configured as an anatomically-shaped graft for subtalar fusion is illustrated. The precision implant 100d can be used, for example, to restore arch and correct valgus deformities during subtalar fusions. In one aspect, the precision implant 100d can be used when a subtalar fusion is required and there is substantial bone loss such that a reduction is necessary to regain the proper length of the limb, for example, when there is a failed fusion and necrotic bone is present and must be removed. The surgical procedure can be performed with a medial approach to the subtalar joint 86 between the calcaneus 80 and the talus 84. The precision implant 100d can be configured to match the footprint of the articulating surfaces 88 being fused. More specifically, the precision implant 100d can be designed to maximize the graft/host interface, as well as match and align the load bearing portion of the precision implant 100d with the cortex of the bone, reducing graft subsidence.

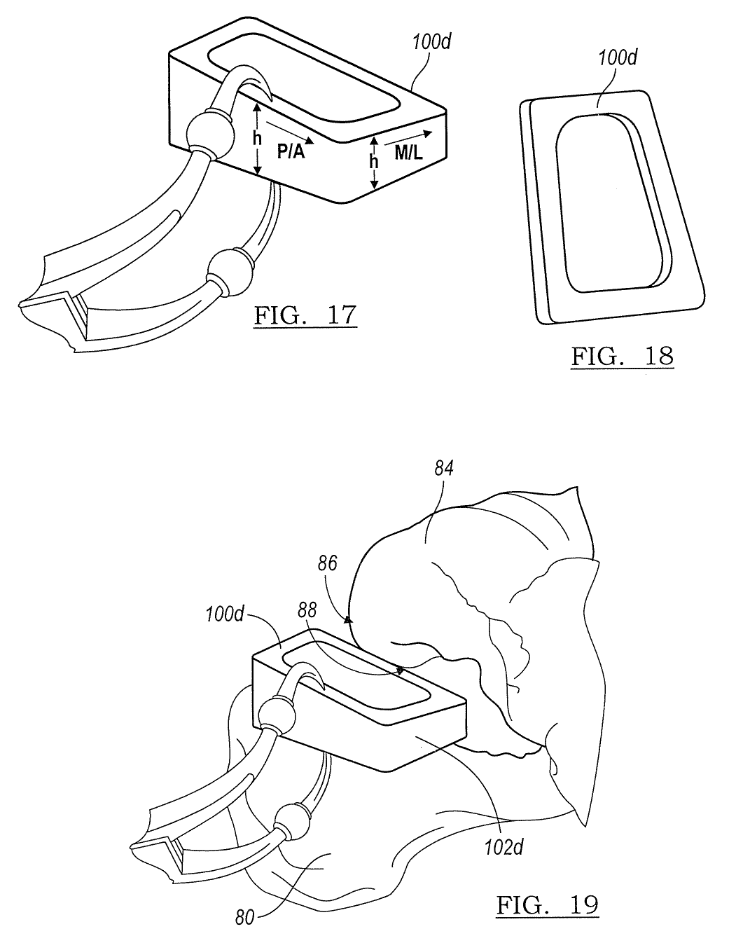

In one aspect, and more specifically, the precision implant 100d can have a parallelepiped shape with trapezoidal cross-section and rounded corners. The precision implant 100d can also define a non-load-bearing central bore 112 for allowing tissue ingrowth. The central bore 112 can be divided by a cross-bar into separate sub-bores. It will be appreciated that additional crossbars 110 can be provided, as desired. In an exemplary aspect, the cross-section of the precision implant 100d can have radii of curvature of about 0.0625 inches, for a length “L” of about 25 mm. The first and second widths W₁, W₂ Of the graft cross-section can be about 14 mm and 23 mm respectively. The graft wall thickness “t” can be about 3 mm, or other thickness chosen for mechanical strength and for generating enough surface area to reduce graft subsidence. The crossbar 110 can provide structural reinforcement during implantation and can be optionally centrally located. The precision implant 100d can have bi-planar tapers along Posterior-Anterior (P/A) and Medial-Lateral (M/L) directions, as illustrated by respective arrows in FIG. 17, to restore the arch and the angle of the foot to their proper position. The P/A taper can be defined, for example by elevations h tapering from about 12 mm to about 9 mm. The M/L taper can be defined by elevations h tapering from about 9 mm to about 6 mm.

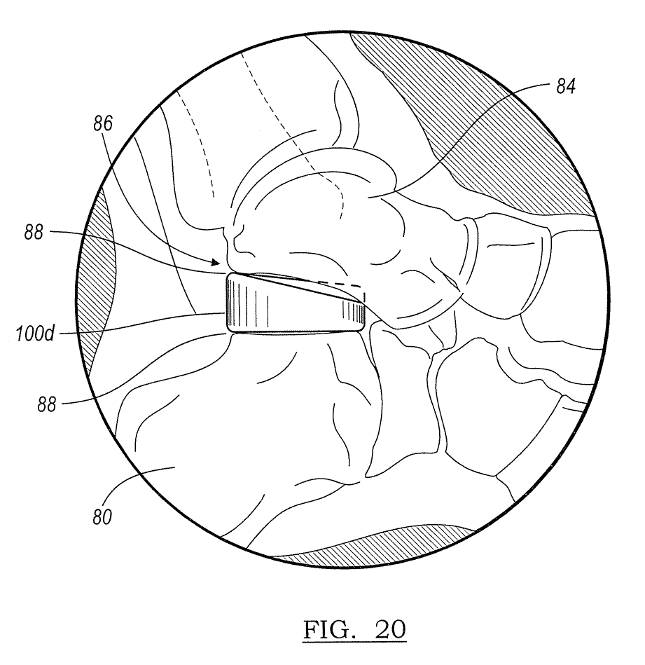

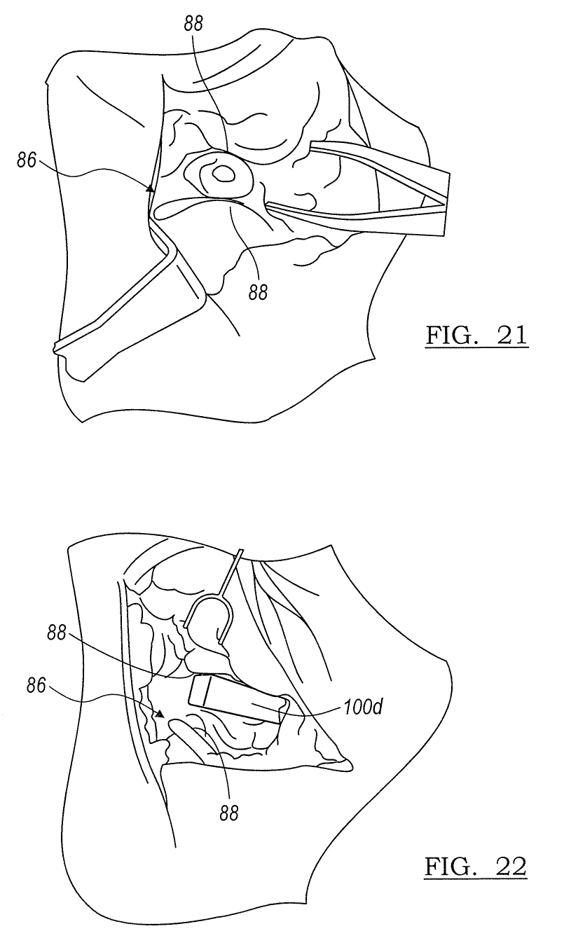

Referring to FIGS. 19 and 21, the articular surfaces 88 of the subtalar joint 86 can be resected. Referring to FIG. 22, the precision implant 100d can be inserted between the resected articular surfaces 88 to maintain anatomical reduction for proper fusion. A drawing of a radiographic view showing the precision implant 100d inserted between the resected articular surfaces 88 is illustrated in FIG. 20.

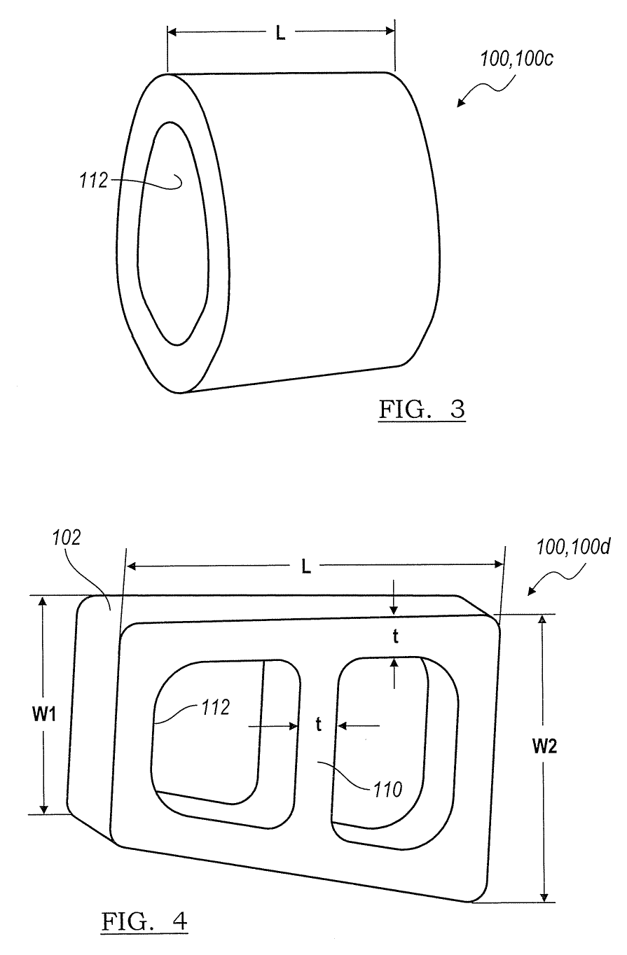

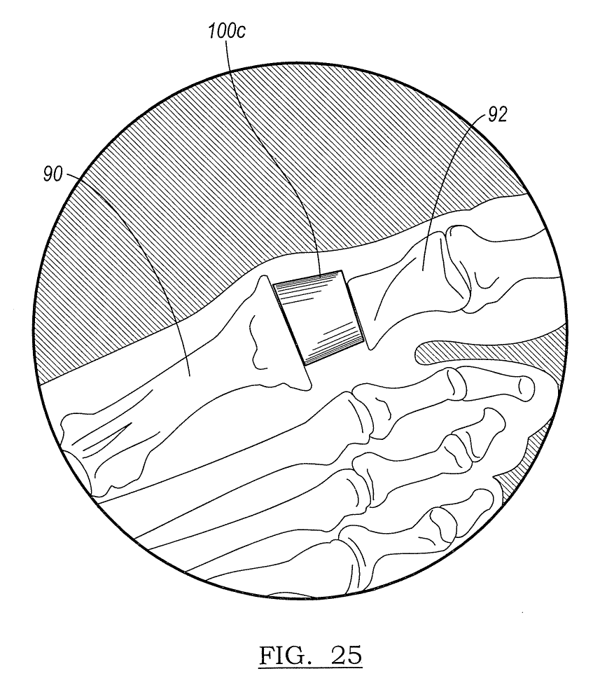

Referring to FIGS. 3 and 23-27, a precision implant 100c configured as an anatomically-shaped graft for hallux metatarsal-phalangeal (MP) fusion is illustrated. In one aspect, the precision implant 100c can be used in hallux MP fusions of the first metatarsal 90 and first phalange 92 when there is substantial bone loss such that a reduction is necessary to regain the proper length of the toe, for example when there is a failed fusion and necrotic bone is present and must be removed. The precision implant 100c can be a designed such that it matches the cross-section of the first metatarsal at the metaphyseal region and tapers, for example, about 1.5 mm in all directions to match the cross-section of the first phalange.

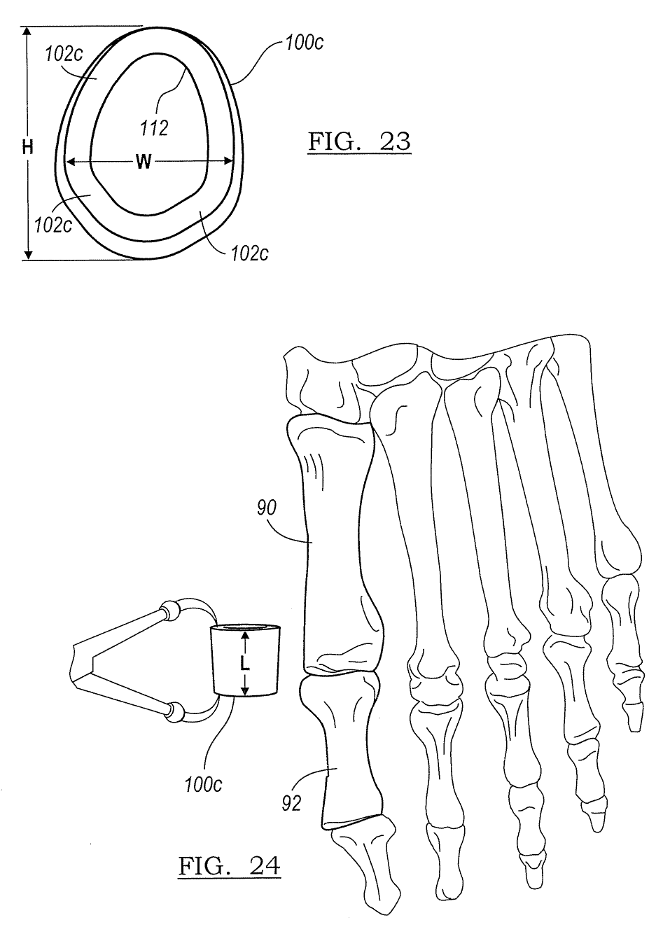

The cross-section of the precision implant 100c can be generally of elliptical or other closed-curve shape. The cross-section of the precision implant 100c can include a central bore non-load-bearing, and can be comprised of a series of arcs 102c of varying radii of curvature, as illustrated in FIG. 23. On the metatarsal side, the overall height “H” of the cross-section can be, for example, about 21 mm, and the overall width “W” of the cross-section can be about 18 mm. On the phalangeal side, the overall height H can be, for example, about 18 mm, and the overall width W of the cross-section about 15 mm. These dimensions and the selections of the arcs 102c that comprise the cross-sectional shape can be chosen such that they will allow the load bearing portion of the precision implant 100c to be lined up with the cortices of the first metatarsal 90 and first phalange 92 to reduce the risks of graft subsidence, which can reduce the effectiveness of the procedure. The wall thickness “t” can be about 2 mm, or other value chosen for mechanical strength and for generating enough surface area to reduce graft subsidence. The graft length “L” can be, for example, about 15 mm.

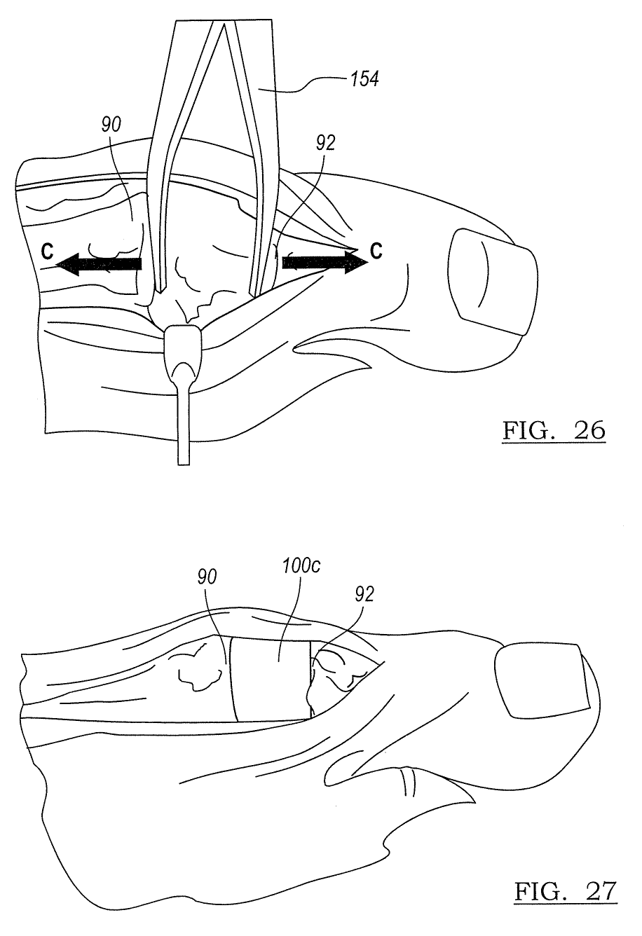

Referring to FIG. 26, the toe can be brought to the correct length by moving the first metatarsal bone 90 and the first phalange bone 92 in the direction of opposite arrows “C”. The precision implant 100c can be then inserted into the MP fusion site to correct the toe length, as illustrated in FIG. 27.

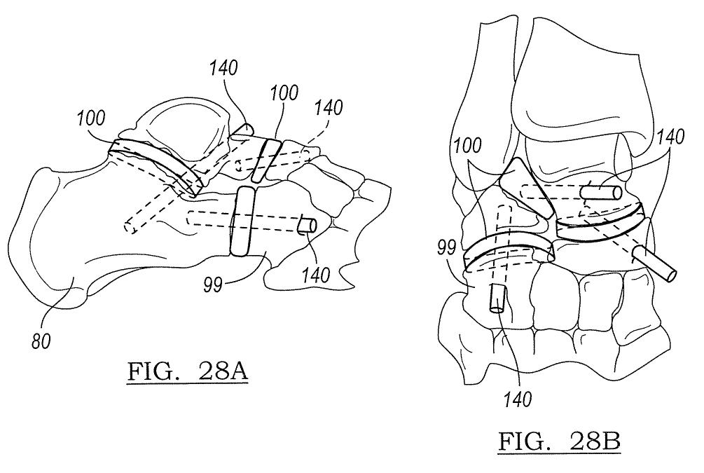

Referring to FIGS. 28A and 28B, it will be appreciated that a particular implant 100 can be optionally secured to adjacent bones 99 by using one or more known fasteners 140 through the central bore 112 of the implant 100.

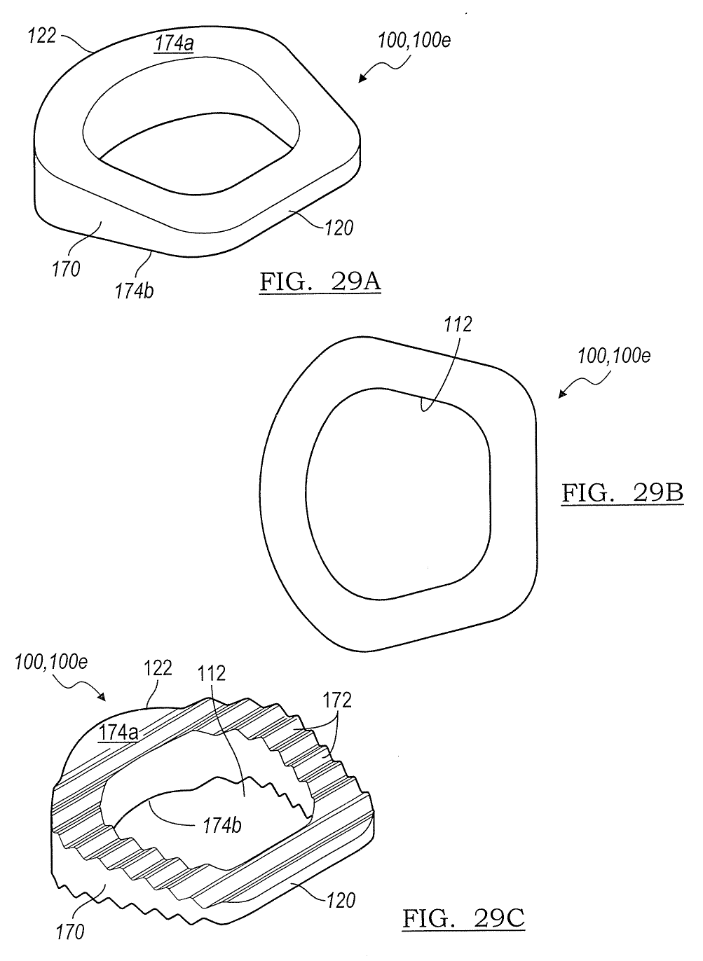

Although various implants 100 for specific conditions of the foot/ankle were illustrated, it will be appreciated that the implants 100 and methods of the present teachings can be applied to other foot/ankle procedures. Referring to FIGS. 29A-C, or example, anatomically configured implants 100e can be used as opening wedges in supramaleolar osteotomy procedures. Supramaleolar osteotomy involves an opening wedge osteotomy of the tibia superior to the ankle for correction of limb deformities, such as club foot. As can be seen in FIG. 29A, the precision implant 100e can have a peripheral wall 170 in the form of wedge tapering from a trailing edge 122 to a leading edge 120. The precision implant 100e can be configured such that the medial-lateral and anterior-posterior cross-sections match the cross section of the distal metaphyseal region of an adult tibia. Referring to FIG. 29C, in one aspect the precision implant 100e can be provided with teeth, ridges or other engagement formations 172 formed on opposite upper and lower faces 174a, 174b for engaging corresponding opposite faces of the tibia to help void implant movement or slippage from the site. It will be appreciated that similar engagement formations 172 can be provided for the other implants 100a-100d, and 100f discussed below.

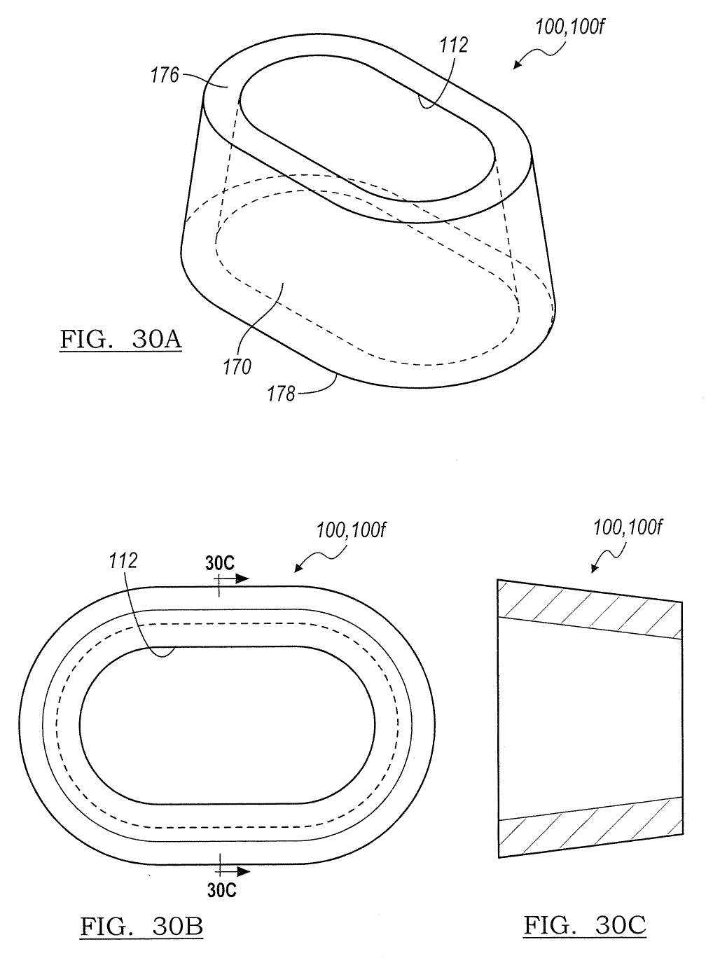

Similarly, anatomically configured precision implants 100 can be used as an ankle fusion spacer 100f in ankle fusions with substantial bone loss resulting from trauma or after a failed total ankle replacement. Referring to FIGS. 30A-C, the precision implant 100f can be designed to match the cross-section of the talus. As seen from FIGS. 30B and C, the precision implant 100f can have a peripheral wall 170 that can taper between opposing faces 176 and 178 in both the medial-lateral and posterior-anterior orientations by several millimeters to fit within the extents of the tibia and fibula. Several sizes can be provided to accommodate bone loss suffered by different bones.

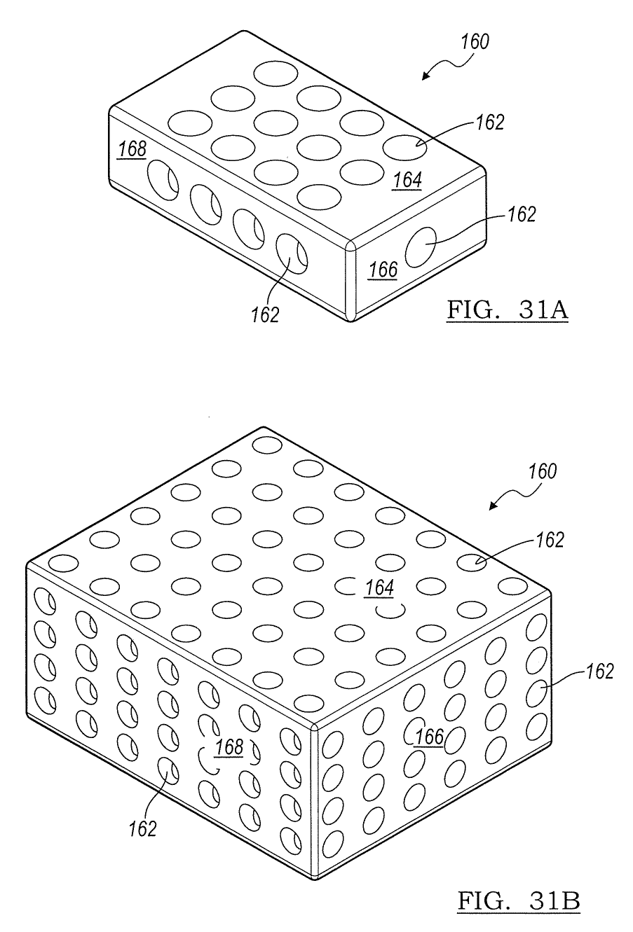

Referring to FIGS. 31A and B, a porous utility block 160 having a network of holes 162 oriented in three orthogonal planes 164, 166, 168 throughout the block can be adapted for shaping into a precision implant 100 at the time of surgery using standard powered surgical equipment, such as osteotomes, burrs, drills, or other instruments. The utility block 160 can be provided in different sizes and with different configurations of holes. FIGS. 31A and B illustrate exemplary utility blocks 160 with representative dimensions 36 mm×30 mm×23 mm and 25 mm×15 mm×11 mm, respectively. The resulting precision implant 100 can accordingly include a three-dimensional network of holes 162.

As discussed above, the precision implants 100a-f can be pre-formed of a resorbable ceramic-polymer composite, such as BioPlex, or provided as utility blocks 160 to be shaped at the time of surgery. Further, any of the elements of each of the precision implants 100a-f can be included in any combination to another precision implant. For example, each precision implant 100 can include one or more crossbars 110 defining one or more bores or sub-bores 112.

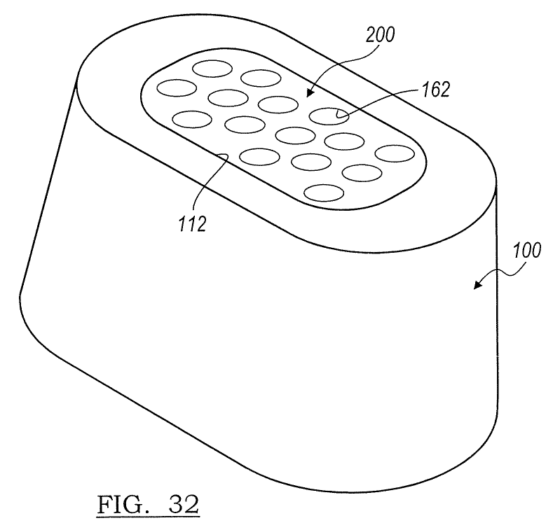

Referring to FIG. 32, a precision implant 100 can include a central bore 112 receiving an insert 200. The insert 200 can be made of a resorbable ceramic-polymer composite, such as BioPlex, or Pro Osteon, or other graft constructs comprising allograft, autograft, synthetic constituent materials, or combinations thereof. The insert 200 can be shaped to conform to the shape of the bore 112. The insert 200 can also include a three-dimensional network of holes 162.

The foregoing discussion discloses and describes merely exemplary arrangements of the present invention. One skilled in the art will readily recognize from such discussion, and from the accompanying drawings and claims, that various changes, modifications and variations can be made therein without departing from the spirit and scope of the invention as defined in the following claims.

What is claimed is:

1. An orthopedic device comprising:
a foot/ankle implant anatomically-shaped for implantation between two bone portions of the foot or ankle to correct associated deformities, the foot/ankle implant having a peripheral wall surrounding a central bore therethrough and defining an annular cross-section, the wall constructed from a composite material, the composite material comprising a ceramic component and a polymer component, the ceramic component gradually resorbable after implantation, and the polymeric component gradually degradable after implantation.

2. The orthopedic device of claim 1, wherein the foot/ankle implant is wedge-shaped for insertion in a calcaneous osteotomy, the foot/ankle implant having a leading edge and a trailing edge, the leading edge having a leading elevation smaller than a trailing elevation of the trailing edge, the annular cross-section having a closed curve perimeter including a plurality of arcs of varying radii.

3. The orthopedic device of claim 2, wherein the foot/ankle implant includes a cross bar dividing the central bore into first and second sub-bores.

4. The orthopedic device of claim 1, wherein the foot/ankle implant is wedge-shaped for insertion in a cuneiform osteotomy, the foot/ankle implant having a leading edge and a trailing edge, the leading edge having a leading elevation smaller than a trailing elevation of the trailing edge, the annular cross-section having a trapezoidal shape.

5. The orthopedic device of claim 4, wherein the foot/ankle implant is configured to conform to the cross-section of the medial cuneiform.

6. The orthopedic device of claim 5, wherein the foot/ankle implant extends approximately two-thirds of the medial cuneiform's depth.

7. The orthopedic device of claim 1, wherein the foot/ankle implant is configured for insertion between resected articulating surfaces of a subtalar joint and comprises a parallelepiped having rounded corners, wherein the annular cross-section and the central bore of the foot/ankle implant are trapezoidal, and wherein the parallelepiped is bi-planarly tapered in posterior/anterior and medial/lateral directions.

8. The orthopedic device of claim 7, further comprising a cross bar dividing the central bore into two sub-bores.

9. The orthopedic device of claim 1, wherein the foot/ankle implant is configured for insertion in metatarsal-phalangeal fusion, the foot/ankle implant tapered in all directions to conform to the cross-sections of the two bone portions, wherein the annular cross-section of the foot/ankle implant is curved and comprises a plurality of arcs of varying radii of curvature.

10. The orthopedic device of claim 1, wherein the foot/ankle implant is anatomically-shaped and configured as a wedge for insertion in a supramaleolar osteotomy.

11. The orthopedic device of claim 1, wherein the foot/ankle implant configured to mate with a cross-section of a talus in ankle fusion, and wherein the peripheral wall is curved and extends between opposing first and second faces, the peripheral wall and the central bore tapering in a medial-lateral orientation and in a posterior-anterior orientation.

12. The orthopedic device of claim 11, further comprising a resorbable insert having a shape conforming to the central bore and received in the central bore.

13. The orthopedic device of claim 12, wherein the insert is a ceramic-polymer composite.

14. The orthopedic device of claim 1, wherein the foot/ankle implant includes opposing bone-engagement faces including grooves, ridges, or teeth for engaging the bone.

15. The orthopedic device of claim 1, wherein the foot/ankle implant is formed from a block defining a three-dimensional network of holes throughout.

16. The orthopedic device of claim 1, further comprising a resorbable insert having a shape conforming to the central bore and received in the central bore.

17. The orthopedic device of claim 16, wherein the insert is formed from a porous material including a three-dimensional network of holes.

18. A method for correcting foot/ankle deformities, the method comprising:
providing a resorbable polymer-reinforced ceramic composite block;
shaping the composite block to an anatomically-shaped and load-bearing foot/ankle implant for implantation between two bone portions of the foot or ankle to correct associated deformities;
maintaining an opening between the two bone portions before inserting the implant; and
inserting the foot/ankle implant in the opening such that the implant substantially matches the cross-section of the bone portions.

19. The method of claim 18, wherein shaping comprises one of pre-operatively shaping or intra-operatively shaping.

20. The method of claim 19, further comprising:
forming a central bore in the implant; and
inserting a resorbable insert into the central bore.
